(19) United States
(12) Patent Application Publication
(10) Pub. No.: US 2004/0022793 A1
(43) Pub. Date: Feb. 5, 2004

(54) Vaccine comprising active agent immunogenic acyl glyceryl phosphatidylinositol manno-oligosaccharide

(76) Inventors: Wayne Bruce Severn, Upper Hutt (NZ); Graham Stephen Le Gros, Wellington (NZ); Jacque Lucille Harper, Lower Hutt (NZ); Paul Henry Atkinson, Lower Hutt (NZ)

Correspondence Address: JACOBSON HOLMAN PLLC, 400 SEVENTH STREET N.W., SUITE 600, WASHINGTON, DC, 20004, US

(21) Appl. No.: 10/311,340

(22) PCT Filed: Jul. 2, 2001
(86) PCT No.: PCT/NZ01/00131

(30) Foreign Application Priority Data
Jul. 3, 2000 (NZ) 505538

(51) Int. Cl. A61K 39/00; A61K 31/715

(52) U.S. Cl. 424/184.1; 514/61

(57) ABSTRACT

The present invention relates to the prophylactic treatment against, or therapeutic treatment of Th2-mediated diseases or disorders, Vaccines useful in these methods of treatment are provided. The vaccines comprise as active agent immunogenic acyl glyceryl phosphatidyinositol manno-oligosaceharide.

This invention relates to the treatment of Th2-mediated diseases or disorders. More particularly, it relates to both therapeutic treatment of patients suffering from such diseases or disorders and to preventative (prophylactic) treatment of non-suffers against such diseases or disorders.

### BACKGROUND ART

There are numerous Th2-mediated diseases and disorders. These include allergic and atopic disorders. Such as allergic rhinitis, dermatitis and psoriasis. Asthma is another example, and is broadly representative of the type of disorder which is the focus of treatment herein.

Asthma is a chronic inflammatory disorder of the airways in which many cells play a role, including mast cells and eosinophils. In susceptible individuals this inflammation causes symptoms which are usually associated with widespread but variable airflow obstruction that is often reversible either spontaneously or with treatment, and causes an associated increase in airway responsiveness to a variety of stimuli.

Asthma can be inherited, is not contagious and may be chronic and persistent or occurring in the form of attacks which are periodic and usually at least partly reversible. Attacks vary in severity and frequency from person to person. Many factors may contribute to the development of asthma including exposure to inhaled allergens such as pollens, mold spores, house dust mites and animal dander. In an individual who has developed asthma, many stimuli can trigger asthma attacks including allergens, viral respiratory infections (colds or the flu), irritants in the air (smoke, air pollution, perfume), damp, cold weather, and exercise.

During an asthma attack, the muscles around the bronchial tubes tighten and the linings of the bronchial tubes swell (become inflamed) and produce thick mucus, thereby decreasing the internal diameter of the tubes. These changes increase resistance to the flow of air making it hard to breathe. When asthma is properly controlled the bronchial tubes are of normal size.

Asthma is a common disease among both children and adults. An estimated 7% of people in the United States have been diagnosed as asthmatic. The corresponding figure for New Zealand is about 10% (Burney, P. et al. (1996) Variations in the Prevalence of Respiratory Symptoms, Self-Reported Asthma Attacks, and Use of Asthma Medication in the European Community Respiratory Health Survey. Eur. Respir. J 9:687-695). The occurrence of asthma in both Western and developing countries has increased markedly over the last 30 years. This relatively short time frame suggests that environmental rather than genetic factors are at work.

In most cases asthma is an atopic disorder in which the underlying process is due to an allergic response to common environmental allergens. This allergic response is a function of the immune system characterised by activation and recruitment of eosinophils to the lung causing the characteristic chronic swelling and inflammation of the airways that affects the breathing of sufferers.

The pharmaceutical treatment of asthma includes several different classes of drugs, including beta agonists, topical or oral steroids and theophyllines. If used appropriately, such treatments may keep asthma systems from developing or relieve them when they are present. Beta agonists and theophyllines primarily act by relaxing the muscles surrounding the airways while steroids act to reduce (and even prevent) inflammation and mucus production. Other medications exist and more are being developed due to the growing interest in and concern over the prevalence, morbidity and mortality of asthma world-wide.

There is an immunological basis to the development of airways inflammation in asthma, involving the Th2 lymphocytes (Th2s). These cells secrete cytokines, including interleukin-4 (gL4) and IL-5, leading to enhanced production of immunoglobulin E (IgE) by B cells and the generation and recruitment of eosinophils respectively. Activation of mast cells by allergens releases histamine and other mediating chemicals that trigger an acute inflammatory response, including mucus production. Eosinophils release mediators including cytotoxins which lead to inflammation and necrosis of the bronchial epithelium. The localised recruitment and activation of eosinophils together with the resultant tissue damage is termed “eosinophilia”.

A need therefore exists for a treatment that modulates the immune system to reduce the risk of developing atopy and airways inflammation, in addition to the traditional treatment with drugs which suppress airways inflammation once it has already occurred, or drugs which reduce symptoms in an asthmatic individual. An added benefit would be if such a treatment also has a similar inhibitory effect in a current sufferer of an atopic disorder to reduce the severity of their disease.

One immunological approach to meet this need involves Mycobacterium bovis—Bacillus Calmette-Guerin (BCG). Prior active infection with this organism has been reported by Erb et at (J. Exp. Med, Vol. 187, No. 4, Feb. 16, 1998) to suppress subsequent allergen-induced airway eosinophilia in mice, with intranasal infection being reported to be more effective than intraperitoneal or subcutaneous infection.

BCG as an organism and as BCG-Polysaccharide Nucleic Acid has also been reported as being used in the treatment of asthma in China (see, for example, China J. Paedia (1991); 39(3): 165-167, Guangzhou Medical Journal 1984; 15(2):16-18) and Acta of Hu-Nan Medical University 1992; 17:365-367. Intact BCG is reported as being administered both alive and dead. The reported routes of administration vary between intramuscular injection and scratch vaccination.

Lipoglycans (including LAM) have been included in immunological compositions previously. For example, U.S. Pat. No. 5,853,737 (Modlin) discusses various methods of inducing a CD1 restricted immune response and teaches of a vaccine containing CD1-presented non-polypeptide hydrophobic antigens and in particular a lipoarabinomannan (LAM) antigen.

Both U.S. Pat. Nos. 4,329,452 and 4,394,502 (Maruyama) teach of the use of lipopolysaccharide as an active component in an immunotherapeutic agent for tumours. The lipopolysaccharide can be derived from human tubercle bacillus.

LAM has also been reported to have efficacy in the suppression of airway eosinophilia. It is suggested to be the component of BCG responsible for the effects of BCG in suppressing allergen-induced airway eosinophilia in mice reported by Erb et al (see above).

The applicants have now surprisingly found that there is an additional active component of the mycobacterial cell wall which is capable of suppressing Th2 mediated responses, and their consequent physical effects such as airway eosinophilia. The present invention is therefore directed to an alternative immunological approach involving this further active agent, acyl glyceryl phosphatidylinositol manno-oligosaccharide (PIM), in immunogenic form.

PIM extracts from M. tuberculosis have been reported to be involved in the recruitment of natural killer T (NKT) cells (Apostolou et al. PNAS, 1999, 96, 5141-6). The use of PIM-activated NKT cells to induce a granulomatous response is taught in Apostolou et al. 2000 and WO 0063348.

Synthetic non-peptide antigens comprised of hydrophobic and hydrophilic components have also been reported (Porcelli and Moody, 1999, WO 99 12562, U.S. Pat. No. 6,236,676). The CD1-restricted proliferation of the T cell line LDN5 in-vitro following treatment with synthetic, and mycobacteria-derived, antigens is described.

However, to the applicant's knowledge, PIM has not been employed or proposed as an immunoactive agent in a vaccine for treating Th2-mediated diseases or disorders, either prophylactically or therapeutically.

It is therefore an object of this invention to provide an immunological approach to the treatment of such diseases and disorders, both prophylactically and therapeutically which at least provides a useful choice over existing approaches.

### SUMMARY OF THE INVENTION

In a first aspect, the invention provides a vaccine for inducing an immune response in a patient effective in the prophylactic treatment against, or therapeutic treatment of, a Th2-mediated disease or disorder which comprises, as active agent, immunogenic acyl glyceryl phosphatidylinositol manno-oligosaccharide (PIM).

As used herein, “immunogenic PIM” means PIM other than as part of an intact mycobacterial organism, which PIM is capable of inducing an immune response in a patient.

As used herein, “prophylactic treatment against a Th2-mediated disease or disorder” means treatment of a non-sufferer from such a disease or disorder to prevent or at least reduce the likelihood of that individual suffering from that disease or disorder.

As used herein, “therapeutic treatment of a Th2-mediated disease or disorder” encompasses preventing, or reducing the severity of or associated with the symptoms of or associated with a Th2-mediated disease or disorder, inclusive of bronchial inflammation and eosinophiha.

Conveniently, the Th2-mediated disease or disorder is selected from allergic and atopic disorders.

Examples include allergic rhinitis, dermatitis and psoriasis. Preferably, said Th2-mediated disease or disorder is asthma.

In a further embodiment, the invention provides a vaccine for inducing an immune response in a patient suffering from or susceptible to a condition which involves bronchial inflammation and/or airway eosinophilia which comprises, as active agent, immunogenic PIM.

Preferably, said vaccine is formulated for respiratory administration to said patient. However, formulations for other routes of administration are also contemplated, these but not being limited to subcutaneous, intradermal, intramuscular, intraurethral, intrarectal, intravaginal and intraoccular.

As used herein, “respiratory administration” means administration to the airways of a patient, including administration intranasally and by inhalation through the mouth to reach the respiratory tract.

The invention further provides a vaccine for reducing the severity of a Th2-mediated disease or disorder comprising an immunologically effective amount of immunogenic PIM. Again, said vaccine is preferably formulated for respiratory administration.

Still further, the invention provides a vaccine for reducing the risk of developing a Th2-mediated disease or disorder comprising an immunologically effective amount of immunogenic PIM, preferably formulated for respiratory administration.

Conveniently, said immunogenic PIM is isolated from a mycobacterium, more conveniently isolated from an M. bovis organism and most conveniently is isolated from M. bovis strain AN5.

It will be usual for said immunogenic PIM to be a fluid, and preferably in the form of a solution or suspension.

Conveniently, where as is preferred the vaccine is for respiratory administration, it will further comprise a respiratorially acceptable adjuvant, which may include a detergent or surfactant component.

A secondary immunogen selected from one or more Th1 type immune response inducing substances may also be present. Preferably, Mycobacterium bovis (Bacillus Calmette-Guerin) is included as said Th1 type immune response inducing substance, although LAM can also be employed as the secondary immunogen.

In another aspect, the invention provides a method of prophylactically treating a patient against a Th2-mediated disease or disorder which comprises the step of inducing an immune response in said patient by administering an effective amount of immunogenic PIM.

Preferably, said PIM is respiratorially administered.

In yet another aspect, the invention provides a method of therapeutically treating a Th2-mediated disease or disorder in a patient which comprises the step of inducing an immune response in said patient by administering an effective amount of immunogenic PIM.

Again, it is preferred that the PIM be respiratorially-administered.

Conveniently, said immunogenic PIM is administered in the form of a vaccine as described above.

Usually, the immunogenic PIM will be administered by inhalation through the mouth or intranasally to said patient.

In yet another aspect, the invention provides the use of immunogenic PIM in the preparation of a medicament for the therapeutic treatment of a Th2-mediated disease or disorder.

In still another aspect, the invention provides the use of immunogenic PIM in the preparation of a medicament for prophylactic treatment against developing a Th2-mediated disorder.

In preferred embodiments, the immunogenic PIM is isolated from a mycobacterium, more preferably an M. bovis organism, and most preferably M. bovis strain AN5.

It will be usual in preparing said medicament that said immunogenic PIM be combined with a respiratorially acceptable adjuvant such that the medicament is formulated for respiratory administration.

In a final aspect, the invention provides a device for prophylactically or therapeutically treating a Th2-mediated disease or disorder which includes a container from which a vaccine as described above can be dispensed to the airways of a patient in need of such treatment.

The device will conveniently be one from which said vaccine is dispensable for inhalation through the mouth of a patient, or intranasally dispensable.

### BRIEF DESCRIPTION OF THE DRAWINGS

FIG. 1 is a graph showing number of cells per ml of BAL exudate. Mice (4-5 per group) were treated with PIM.

FIG. 2 is a graph showing percentage of eosinophils recovered by BAL. Mice (4-5 per group) were treated with PIM.

FIG. 3 is a graph showing number of eosinophils per ml recovered by BAL. Mice (4-5 per group) were treated with PIM.

FIG. 4 is a graph showing the number of eosinophils per ml recovered by BAL. Mice (4-5 per group) were treated with deacylated PIM.

FIG. 5 is a graph showing the number of eosinophils per ml recovered by BAL. Mice (4-5 per group) were treated with PIM isolated from M. smegmatis.

FIG. 6 is a graph showing the number of eosinophils per ml recovered by BAL. Mice (4-5 per group) were treated with PIM 1 week following the second i.p. injection and 6 weeks before OVA challenge.

FIG. 7 is a graph showing the number of eosinophils per ml recovered by BAL. Mice (4-5 per group) were treated with PIM between 8 and 2 weeks before OVA challenge. OVA i.p. sensitisation was at 4 and 2 weeks.

FIG. 8 is a graph showing the number of eosinophils per ml recovered by BAL. Mice (4-5 per group) were treated with PIM at the same time as the OVA challenge.

FIG. 9 is a graph showing the number of eosinophils per ml recovered by BAL in CD1 knockout mice. Mice (4-5 per group) were treated with PIM.

FIG. 10 is a graph showing the number of eosinophils per ml recovered by BAL in IFNγ knockout mice. Mice (4-5 per group) were treated with PIM.

### BEST MODE OF PERFORMING THE INVENTION

As broadly outlined above, the present invention offers an approach to treating a Th2-mediated disease or disorder in a patient. This makes the invention particularly applicable to the treatment of asthma in an asthmatic and/or for reducing the risk of developing airway eosinophilia and thus asthma in a non-asthmatic.

Other immune and/or atopic disorders to which the invention has application include allergic rhinitis, dermatitis and psoriasis, although these are but examples.

The essential feature of the approach of the invention is the administration of biologically active amounts of acyl glyceryl phosphatidylinositol manno-oligosaccharide (PIM) in an immunogenic form. This is preferably achieved by introduction of PIM to the airways of a patient, but is no way limited thereto. Alternative routes of administration can equally be employed, with transmucosal, intraural, subcutaneous, intradermal, intramuscular, oral, intraurethral, intrarectal, intravaginal and intraoccular being other examples.

By the term “PIM” as used herein what is meant an acyl glyceryl phosphatidyl inositol manno-oligosaccharide which may be LM containing up to 40 mannose units, but which is not LAM.

The fatty acid component comprises one or more fatty acid units, preferably 2 to 6 units, and more preferably 2 units. The fatty acid units are preferentially 10 to 22 carbon atoms in length, and more preferably 16 to 20 carbon atoms in length. The fatty acid component can, for example, be myristate, palmitate, heptadecanoate, stearate, tuberulostearate or linooleneate, or mixtures of these.

PIM's used in the present invention may have the following general formula:

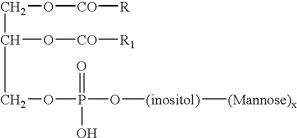

wherein X is 1 to 40, preferably 1 to 6, and R and R₁ independently represent a fatty acid chain.

PIM's used herein may be synthetic or obtained from natural sources. PIM's may be chemically synthesised by reacting a phosphotidyl inositol group with a diacylgycerol, followed by mannosylation or by other methods also known in the art.

PIM is also present in actinomycetes, which are a distinctive lineage of Gram-positive bacteria. Members of this lineage include Rhodococcus equi, Corynebacterium dihtheriae, Corynebacterium matruchotii, Gordona rubropertincta, Gordona terrae, Rhodococcus rhodnii and Tsukamurella paurometabolum.

Other members of the lineage include mycobacteria, with PIM being a component of the mycobacterial cell wall.

For use in the present invention, forms of PIM can therefore also be obtained by isolation from any suitable actinomycetes organism. It is however preferred that the immunogenic PIM for use in the invention be obtained from mycobacteria, particularly pathogenic mycobacteria, or from attenuated strains of pathogenic mycobacteria. However, PIM from non-pathogenic avirulent mycobacteria is by no means excluded.

Particularly suitable mycobacteria from which PIM can be obtained are M. bovis, M. tuberculosis and M. paratuberculosis, with M. bovis organisms such as M. bovis strain AN5 being presently preferred.

The PIM can be isolated from such bacteria, and in particular from mycobacteria, using techniques which are standard in the art. By way of example, the procedure of Severn et al., J. Microb. Methods, 28, 123-30 (1997) can be employed.

Isolated PIM will conveniently be purified for use in the present invention. The effect of this will be to exclude other bacterial components (including bacterial nucleic acid) from the PIM. Again, art standard techniques can be employed such as those described by Severn et at.

Once the PIM is obtained and preferably purified, it is formulated for administration. The detail of formulation will be dependent upon the route of administration chosen, and will be a matter of routine choice for the art-skilled worker.

Preferably, the PIM is formulated for respiratory administration. Respiratory administration requires delivery of the PIM to the airways of the patient to be treated. Generally, this will involve delivery through the mouth or intranasally. Often, inhalation by the patient will provide the motive force to the PIM. However, respiratory administration can also involve delivery by propellant, including in the form of an aerosol generated using a jet or ultrasonic nebuliser. This is presently preferred.

For such applications, the PIM will conventionally be in a fluid form. This can be as a powder or as a solution or suspension particularly for aerosol application).

The PIM will generally also be formulated for respiratory administration together with a respiratorially acceptable adjuvant. The selection of the adjuvant will be dependent upon the formulation and mode of dispensing involved, but will in any case be a matter of routine choice for the skilled worker in this field.

Where, as is preferred, the PIM is to be administered via a nebuliser-generated aerosol, the PIM will be in the form of a solution or suspension which will contain such adjuvant components. One such optional but preferred component is a nontoxic detergent or surfactant. Examples include a Polysorbate 80, beractant (Survanta Susp (Abbott)) and colfosceril palmitate (Exosurf Neonatal (Glaxo Wellcome)).

It is also possible to include an additional immunogen in the solution or suspension for administration as an aerosol. Such an immunogen will generally be a Th1 type immune response inducing substance. One such substance which can be included is BCG, alive or dead, but with dead being preferred. Another such substance is LAM.

Where BCG is included as a secondary immunogen, it will be usual for the solution or suspension to further comprise a non-clumping agent (such as Bovine Serum Albumin) to prevent the organisms from adhering together.

Despite the preference for aerosol administration, it is by no means intended to exclude administration of PIM in other forms. To the contrary, the PIM vaccine can be formulated for administration as a powder, for example using lactose capsules as a delivery vehicle in a dry powder inhaler.

It will also be appreciated that PIM can be used in combined therapy, or formulations, with other therapeutically acceptable medicaments.

The invention will now be exemplified through reference to the following experimental section, which it will be appreciated is illustrative and not limiting.

### EXPERIMENTAL

### Section A

Isolation of Acyl Glyceryl Phosphotidylinositol Manno-oligosaccharides (PIM) from Mycobacterium bovis AN5.

Isolation and Purification

General methods. Triton X-114, proteinase K, RNase and DNase were from Boehringer Mannheim. Commercial reagents and solvents were analytical grade. All experiments were done with MilliQ water. The gel filtration properties of the eluted materials were expressed in terms of their distribution coefficients, Kₐᵥ=(Vₑ−V₀)/(Vt−V₀), where V₀ is the void volume of the system, Vₑ is the elution volume of the specific material, and Vₜ is the total volume of the system.

Bacterial cell culture. M. bovis AN5 was obtained from Central Veterinary Laboratories, Weybridge, U.K. and was grown for eight weeks as pellicles on modified Reids synthetic medium (S. Landi, in G. P. Kubica, and L. G. Wayne (Eds.), The Mycobacteria—A Sourcebook: Production and Standardization of Tuberculin, Marcel Dekker Inc., New York, (1984), pp 505-535). Cells were killed by heating to 100° C. for three hours before being harvested on coarse Whatman filter paper.

Isolation and purification procedures. Collected cells were delipidated by stirring with methanol/chloroform (1:1) and after recovery by centrifugation, the pellet was dried. The then cells were twice washed with 2.5% Tris buffered saline (TBS) (0.05 M, pH 7.5), recovered by centrifugation (10,000 g, 30 min) and freeze dried. Approximately 2 g (dry weight) of cells were slurried in TBS (4 ml) containing EDTA (5 mM) and sodium azide (0.05%), cooled to 4° C. and extruded by passing through a French press twice at 40,000 kPa. The solution of disrupted cells was made up to a volume of 40 ml with TBS and after the addition of MgCl (10 mM) the disrupted cells were digested with RNase and DNase (1 μg ml⁻¹) at 37° C. for 60 min then 60° C. for an additional 60 min.

Triton X-114 was added to the lysed cells to a concentration of 8% (v/v) and after cooling on ice, the solution was stirred at 4° C. for 16 h. The cellular debris was removed by centrifugation (10,000 g, 4° C., 30 min) and the supernatant was incubated at 37° C. to induce phase separation. The lower Triton X-114 rich phase was recovered after centrifugation (4000 g, 30° C., 20 min) and the upper aqueous layer was mixed with the cellular debris and re-extracted as described above. The detergent phases were combined and the lipoglycan was precipitated by the addition of cold ethanol (−20° C., 95%, 5 vol.) and collected by centrifugation (10,000 g, 30 min).

The crude lipoglycan extracts were dissolved in water (10-20 mg ml⁻¹), by stirring overnight, and ultracentrifuged at 35,000 g for 16 hours. The pellets were collected, dissolved in a minimal amount of water and treated with Proteinase K (1 mg ml⁻¹) for one hour at 37° C. then an additional hour at 60° C. The solution was ultracentrifuged twice more, reconstituted in water and lyophilized.

Fractionation procedure. Crude samples were prepared for column chromatography by resuspension in Tris-deoxycholate buffer (Tris-HCl 10 mM, pH 8.0, EDTA 10 mM, NaCl 0.2 M, deoxycholate 0.25%, NaN₃ 0.02%) to a concentration of 10 mg ml⁻¹. The sample was applied to a column (1.5×100 cm) of Sephacryl S-200 (Pharmacia) and eluted using the same buffer. The eluent was continuously monitored for changes in refractive index and fractions (2 ml) were collected and analyzed colourimetrically for neutral glycose (Dubois, M., Gilles, K. A., Hamilton, J. K., Rebers, Pa. and Smith, F. (1956) Colorimetric method for the determination of sugars and related substances. Anal. Chem. 28, 350-356) and by SDS-PAGE. Analysis of the fractions by SDS-PAGE identity the high-molecular-weight species (Kₐᵥ=0.1) as LAM and fractions corresponding to LM and PIM had K_{av's} of 0.3, and 0.8, respectively. The appropriate LAM, LM and PIM fractions were pooled, desalted by ultrafiltration in a centriplus concentrator (Amicon) using a 3000 MW cut-off membrane, resuspended in water and lyophilized.

Fractions containing the LAM, LM and PIM were collected and lyophilized. Pure LAM accounted for approximately 25% of the crude material applied to the column and a total yield of 1.4% of the initial bacterial dry weight. Recoveries of pure LM and PIM after fractionation were 1.0% and 3.7% respectively.

Deacylation of PIM

PIM was suspended in anhydrous hydrazine (30 minutes), cooled and quenched with cold acetone (−70° C.) to destroy excess hydrazine and precipitate the deacylated PIM.

The deacylated PIM was pelleted by centrifugation and the pellet washed with acetone, dissolved in water and lyophilised.

NB. Analysis was as for carbohydrate analysis for the whole PIM molecule.

Analysis of Lipopolysaccharide

The purity of the combined PIM fractions was investigated. PIM was deemed pure based on the following criteria: 0% protein as indicated by the BCA protein assay, absence of nitrogen as indicated by elemental analysis of the purified extracts, the absence of ribose or deoxyribose in the glycose analysis.

The purified PIM was hydrolysed and acetylated by known methods and the resulting mixture of saccharides analysed by GLC.

Carbohydrate and Fatty acid composition of acyl glyceryl
phosphatidylinositol manno-oligosaccharides from M. bovis AN5.
PIM analysis (M. bovis AN5)

| Fatty Acids | Wt % (fatty acids) | Carbohydrate | Molar Ratio |
|---|---|---|---|
| Pentadecanoic | 2.8 | Mannose | 4.7 |
| Hexadecanoic | 49.8 | Arabinose | 0 |
| Hexadecanoic | 1.2 | Inositol | 1.0 |
| 14 Methyl | | | |
| Heptadecanoic | 9.2 | | |
| Heptadecanoic | 2.1 | | |
| 16 Methyl | | | |
| Octadecenoic | 0.9 | | |
| Octadecanoic | 11.9 | | |
| Octadecanoic | 21.7 | | |
| 10 Methyl | | | |
| Nonadecanoic | 0.4 | | |
| 10 methyl | | | |
| Eicosanoic | Trace | | |

### Section B

Immunology Experimental

Model

An ovalbumin (OVA) induced airway eosinophilia mouse model of atopic airway inflammation was used to determine the effectiveness of the immunogenic PIM suppressing the development of airway eosinophilia. This model is widely used to establish “asthma-like effects” in mice—see for example, Erb et al., J. Exp. Med. 187(4):561-569 (1998); Herz et al., J. Allergy and Clinical Immunology, 102:867-874 (1998); and Randaolf et al., J. Clinical Investigation, 104:1021-1029 (1999).

Mice

C57B1/6J mice were bred and housed at the Wellington School of Medicine Animal Facility (Wellington, New Zealand). The experimental procedures were approved by the animal ethics committee and were in accordance with University of Otago (Dunedin, New Zealand) guidelines for care of animals.

OVA-induced Airway Inflammation:

Sensitisation—6-8 week old mice (4-5 mice per group) were injected intraperitoneally (i.p.) with 2 μg ovalbumin (Sigma Chemical Co., St Louis, Mo.) in 200 μl alum adjuvant (Al(OH)₃, Serva, Heidelberg, Germany) at day 0. A booster intraperitoneal injection of 2 μg ovalbumin in 200 μl alum adjuvant was administered at day 14.

OVA challenge—14 days following the second i.p. injection, mice were anaesthetised by a mixture of Ketaoine and Xylazine (Sigma Chemical Co.). The mice were then inoculated intranasally with 50 μl of 2 mg/μl ovalbumin in PBS.

Immunisation Protocols with PIM—

(a) 7 days following the second i.p. injection, mice were anaesthetised as above. The mice were then immunised intranasally with the indicated concentrations of PIM in 50 μl of PBS. Control mice were given PBS intranasally. The mice were challenged intranasally with OVA 7 days following immunisation with PIM.

(b) Mice were immunised as in (a). The OVA intranasal challenge was administered 6 weeks after treatment with PIM.

(c) Mice were immunised intranasally with PIM at 8 weeks, 6 weeks, 5 weeks, 4 weeks and 2 weeks before the intranasal challenge with OVA. Sensitisation of the mice, as described above, occurred at weeks 4 and 2.

(d) Mice were immunised intranasally with PIM at the same time as the intranasal OVA challenge.

Measurement of Airway Eosinophilia:

4 days after intranasal airway challenge with OVA the mice were sacrificed. The trachea was cannulated and bronchoalveolar lavage (BALS) was performed (3×1 ml PBS). Total BAL cell numbers were counted and spun onto glass slides using a cytospin. Percentages of eosinophils, macrophages, lymphocytes and neutrophils were determined microscopically using standard histological criteria.

Results

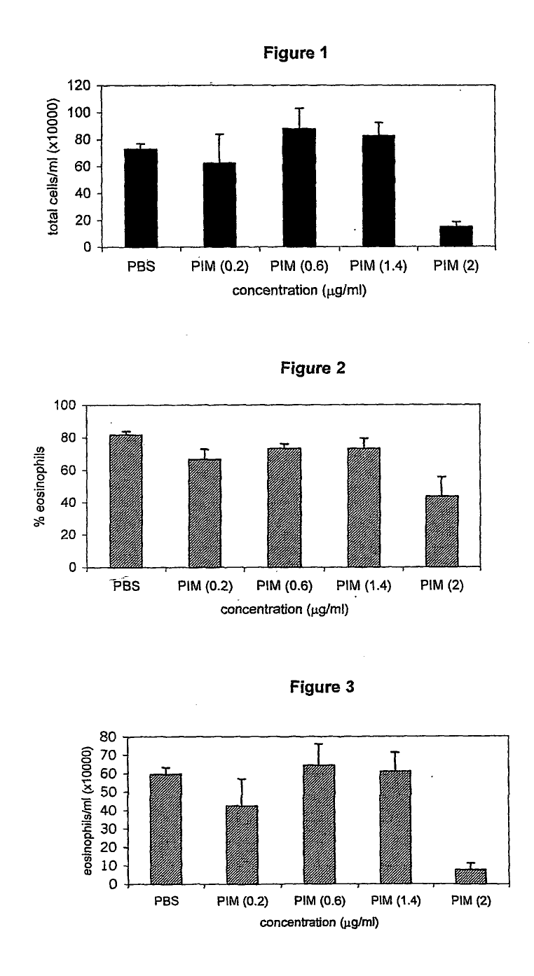

FIGS. 1-10 show the results of the experiments described.

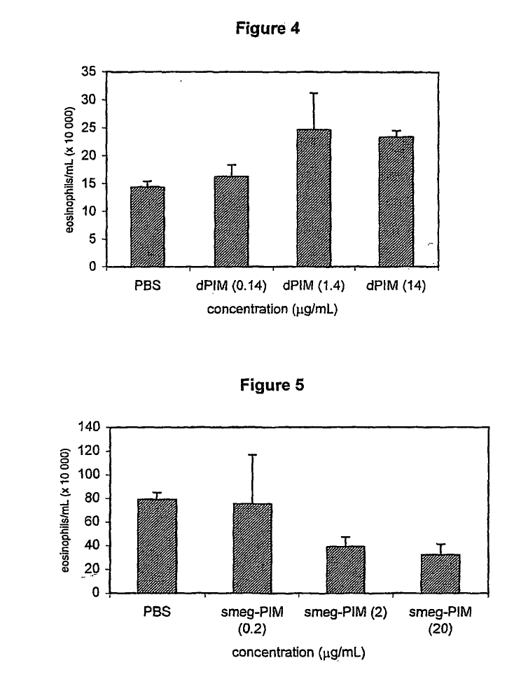

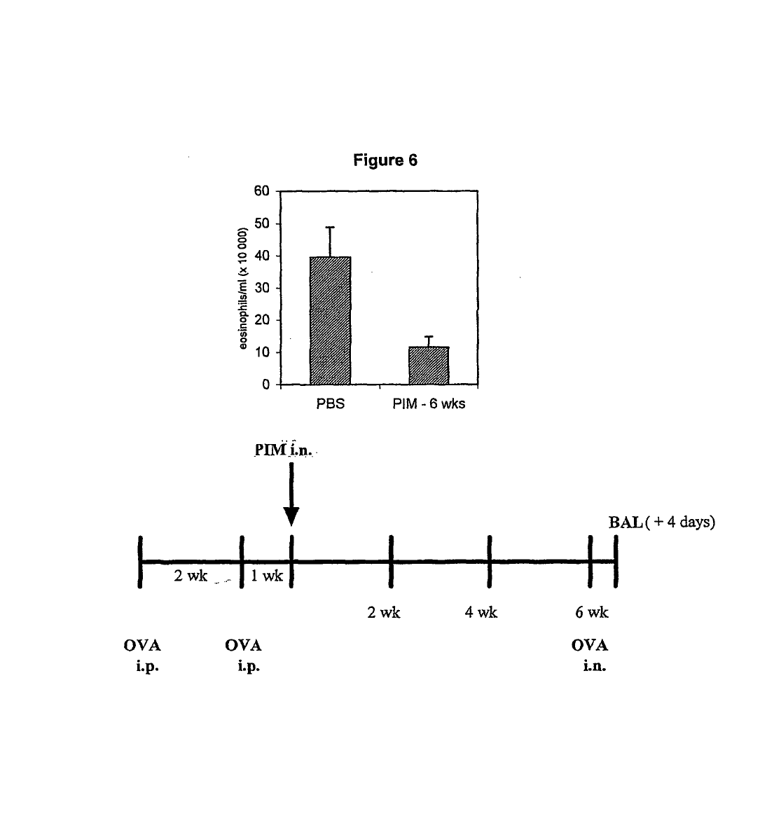

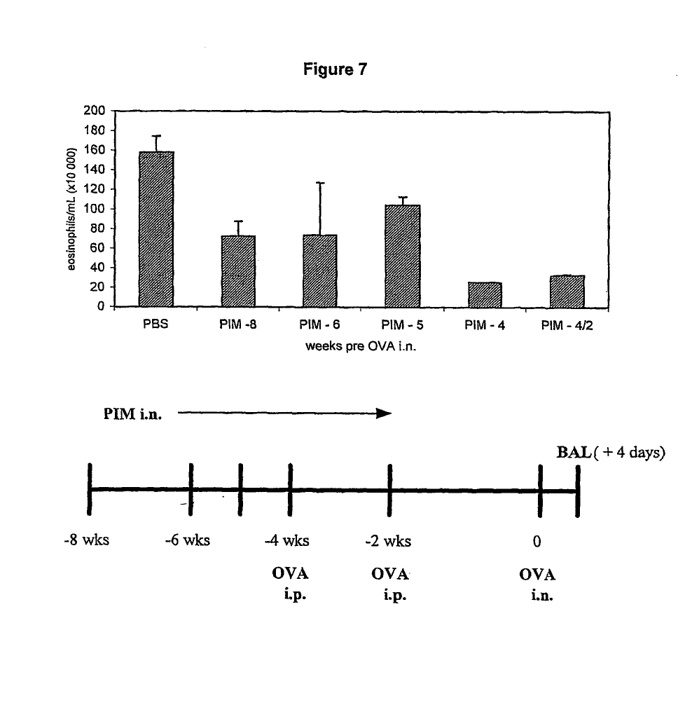

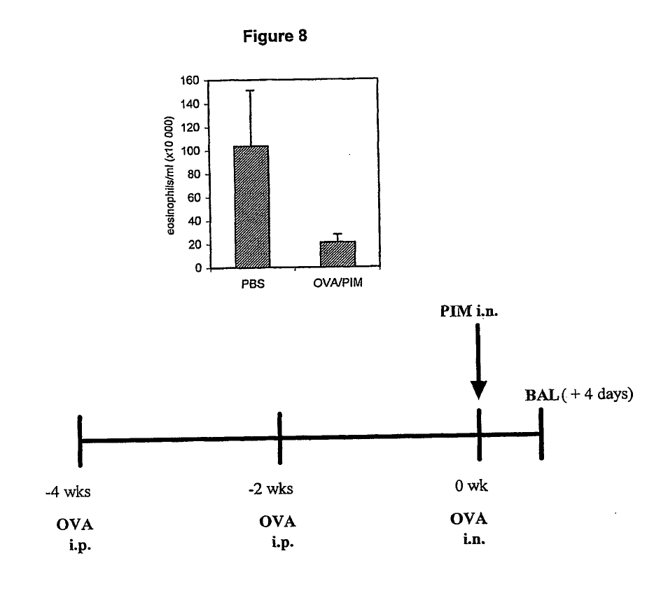

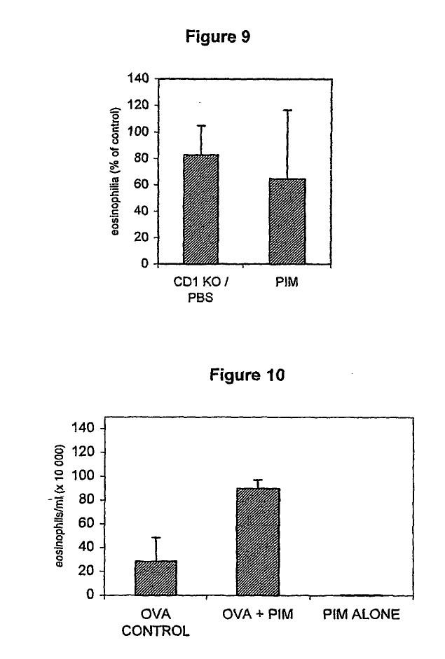

FIG. 1 shows the total number of cells recovered from BAL exudate in mice treated with PIM. FIG. 2 shows the dose-dependent decrease in the percentage of eosinophils in the BAL exudate. FIG. 3 shows the dose-dependent decrease in the number of eosinophils per ml in mice treated with PIM. FIG. 4 shows the effect of deacylated PIM on the number of eosinophils in BAL exudate. FIG. 5 shows the dose-dependent decrease in the number of eosinophils in BAL exudate from mice treated with PIM from non-pathogenic M. smegmatis. FIG. 6 shows the long term suppressive effect of PIM on eosinophils in BAL exudate after sensitisation with OVA. FIG. 7 shows the decrease in the number of eosinophils in BAL exudate from mice treated with PIM before and during sensitisation with OVA. FIG. 8 shows the decrease in the number of eosinophils in BAL exudate from mice treated with PIM at the same time as the OVA challenge. FIG. 9 shows the effect of PIM on the number of eosinophils in BAL exudate in CD1 knockout mice. FIG. 10 shows the effect of PIM on the number of eosinophils in BAL exudate in IFNγ knockout mice.

Conclusion

PIM obtained from pathogenic and non-pathogenic bacteria is efficacious in the suppression of airway eosinophilia. The suppression of eosinophilia can be achieved before, during and after sensitisation to antigen as well as during antigen challenge. These data illustrate a clear application of PIM as an active agent of a vaccine for treating a range of Th2-mediated diseases or disorders, with asthma being a specific example. It is envisaged from the data that PIM could be utilised in both prophylactic and therapeutic application. The suppression of eosinophilia is abrogated by either the removal of the fatty acid tail, the absence of CD1 or IFNγ. It is therefore expected that all three are important in the mechanism of action of the PIM molecule.

### INDUSTRIAL APPLICATION

As will be appreciated from the above, the primary application of the invention is in the treatment of Th2-mediated diseases or disorders. That treatment may be prophylactic, to prevent or reduce the risk of developing such diseases or disorders, or therapeutic, to suppress established disease or symptoms.

The PIM-containing vaccines of the invention are formulated for administration, which will preferably involve respiratory administration by the intranasal or inhaled route for convenience. The inhalation mode of administration will involve the use of a dispensing device, of which a container of PIM vaccine forms a part. That device can be a nebuliser, particularly a jet nebuliser such as that known as the Omron CX (Omron Healthcare, Singapore), the Medic Aid Ventstream or the Wright nebuliser (Aerosol Medicals, Colchester, UK) (where the vaccine is to be administered as an aerosol) or a dry powder inhalation device (such as the devices known as the Accuhaler and Diskhaler (Glaxo Wellcome)).

Respiratorially administered PIM has shown significant efficacy in reducing eosinophil numbers and in turn in reducing bronchial inflammation. The implications of this in both resisting the onset, and reducing the severity, of an Th2mediated condition (such as an asthma episode), and in treating individuals against developing Th2-mediated conditions such as asthma will be apparent to those skilled in this art.

Having described preferred methods of putting the invention into effect, it will be appreciated that modifications can be effected and yet still come within the general concept of the invention. It is to be understood that all such modifications are intended to be included within the scope of the present invention.

1. A vaccine for inducing an immune response in a patient effective in the prophylactic treatment against, or therapeutic treatment of, a Th2-mediated disease or disorder which comprises as active agent, immunogenic acyl glyceryl phosphatidylinositol manno-oligiosaccharide (PIM), in immunogenic form.

2. A vaccine for inducing an immune response in a patient suffering from or susceptible to a condition which involves bronchial inflammation and/or airway eosinophilia which comprises, as active agent, immunogenic PIM.

3. A vaccine for reducing the risk of developing a Th2-mediated disease or disorder comprising an immunologically effective amount of immunogenic PIM.

4. A vaccine according to any one of claims 1-3 wherein said vaccine is formulated for respiratory administration.

5. A vaccine according to any one of claims 1-4 in which said immunogenic PIM is isolated from a mycobacterium.

6. A vaccine according to claim 5 in which said immunogenic PIM is isolated from a M. bovis organism.

7. A vaccine according to claim 6 in which said M. bovis organism is M. bovis strain An5.

8. A vaccine according to any one of claims 1-7 wherein said PIM contains, as the mannose component, from 1-6 mannose units.

9. A vaccine according to any one of claims 1-8 wherein said PIM contains from 2-6 fatty acid units

10. A vaccine according to claim 9 wherein said PIM contains two fatty acid units.

11. A vaccine according to claim 9 or claim 10 wherein said fatty acids are 10 to 22 carbon atoms in length.

12. A vaccine according to claim 22 wherein said fatty acids are 16 to 20 carbon atoms in length.

13. A vaccine according to any one of the preceding claims in which said immunogenic PIM is a fluid.

14. A vaccine according to any one of the preceding claims which further comprises a respiratorily acceptable adjuvant.

15. A vaccine according to any preceding claim which further comprises a secondary immunogen selected from one or more Th1-type immune response inducing substances.

16. A vaccine according to claim 15 in which Mycobactertum bovis bacillus Calmette-Guerin) is included as said Th1 type immune response inducing substance.

17. A method of therapeutically treating a Th2-mediated disease or disorder in a patient which comprises the step of inducing an immune response in said patient by administering an effective amount of immunogenic PIM.

18. A method of therapeutically treating asthma in a patient which comprises the step of inducing an immune response in said patient by administering an effective amount of immunogenic PIM.

19. A method as claimed in claim 17 or claim 18 wherein the said immunogenic PIM is respiratorily administered.

20. A method as claimed in any one of claims 17-19 in which said immunogenic PIM is administered in the form of a vaccine as claimed in any one of claims 1-12.

21. A method according to any one of claims 17-20 in which said immunogenic PIM-is administered by inhalation through the mouth of said patient.

22. A method according to any one of claims 17-20 in which said immunogenic PIM is administered in intranasally to said patient.

23. The use of immunogenic PIM in the preparation of a medicament for the therapeutic treatment of a Th2-mediated disease or disorder.

24. The use of immunogenic PIM in the preparation of a medicament for prophylactic treatment against developing Th2-mediated disorder.

25. A use according to claim 23 or 24 wherein the Th2-mediated disorder is asthma.

26. A use according to any one of claims 23-25 in which said immunogenic PIM is isolated from a mycobacterium.

27. A use according to claim 26 in which said mycobacterium is an M. bovis organism.

28. A use according to claim 27 in which said M. bovis organism is M. bovis strain An5.

29. A use according to any one of claims 23-28 wherein said immunogenic PIM contains as its mannose component, from 1-6 mannose units.

30. A use according to any one of claims 23-29 wherein in preparing said medicament said immunogenic PIM is combined with a respiratorily acceptable adjuvant such that the medicament is formulated for respiratory administration.
